(19) United States
(12) Patent Application Publication
(10) Pub. No.: US 2010/0280325 A1
(43) Pub. Date: Nov. 4, 2010

(54) RETRACTORS AND SURGICAL SYSTEMS INCLUDING THE SAME

(76) Inventors: Tamer Ibrahim, Pleasant Hill, CA (US); Raymond S. Bertolero, Danville, CA (US)

Correspondence Address: HENRICKS SLAVIN AND HOLMES LLP;SUITE 200, 840 APOLLO STREET, EL SEGUNDO, CA, 90245, US

(21) Appl. No.: 12/433,801

(22) Filed: Apr. 30, 2009

(51) Int. Cl. A61B 1/32

(52) U.S. Cl. 600/206

(57) ABSTRACT

Retractors and surgical systems that include retractors are disclosed.

### BACKGROUND

1. Field of the Inventions

The present inventions relate generally to retractors and surgical systems that include retractors.

2. Description of the Related Art

Some surgical systems include flexible articulating arms that may be mounted on a support structure, such an operating table rail, and carry a retractor. The arm allows the surgeon to position the retractor against a tissue surface. Examples of such surgical systems are presented in U.S. Pat. No. 6,860,668 and U.S. Patent Pub. No. 2005/0226682 A1. The present inventor has determined that the retractors associated with conventional surgical systems are susceptible to improvement.

### SUMMARY

A retractor apparatus in accordance with various implementations of at least some of the present inventions includes a malleable retractor. Surgical systems in accordance with various implementations of at least some of the present inventions includes an arm and a malleable retractor that is operably connected to the arm. Because they are malleable, such retractors may be bent into shapes that are suitable for various surgical procedures.

A retractor apparatus in accordance with various implementations of at least some of the present inventions includes a retractor with a relatively hard inner portion and a relatively soft outer portion. Surgical systems in accordance with various implementations of at least some of the present inventions includes an arm and a retractor, with a relatively hard inner portion and a relatively soft outer portion, that is operably connected to the arm. The inner portion of the retractor provides structural stability, while the outer portion makes the retractor atraumatic to tissue.

A retractor apparatus in accordance with various implementations of at least some of the present inventions includes a retractor with first and second sides and a relatively high friction outer surface associated with the first side and a relatively low friction outer surface associated with the second side. Surgical systems in accordance with various implementations of at least some of the present inventions includes an arm and a retractor, defining first and second sides and including a relatively high friction outer surface associated with the first side and a relatively low friction outer surface associated with the second side, that is operably connected to the arm. The relatively high friction outer surface reduces the likelihood that the tissue being retracted will slide relative to the retractor, while the relatively low friction outer surface allows objects, such as other tissue structures and the hands of the surgeon(s) and surgical assistants, to slide past the retractor.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a perspective view of a surgical system in accordance with one embodiment of a present invention.

FIG. 2 is a plan, partial cutaway view of a retractor apparatus in accordance with one embodiment of a present invention.

FIG. 2A is a section view taken along line 2A-2A in FIG. 2.

FIG. 3 is another plan, partial cutaway view of the retractor apparatus illustrated in FIG. 2.

FIG. 4 is a plan view of a retractor apparatus in accordance with one embodiment of a present invention.

FIG. 5 is a section view taken along line 5-5 in FIG. 4.

FIG. 6A is a plan view of a retractor apparatus in accordance with one embodiment of a present invention.

FIG. 6B is a plan view of a retractor apparatus in accordance with one embodiment of a present invention.

FIG. 7 is a plan view of a retractor apparatus in accordance with one embodiment of a present invention.

FIG. 8 is a section view of a linkage assembly in accordance with one embodiment of a present invention.

FIG. 9 is a section view of a portion of a linkage assembly in accordance with one embodiment of a present invention.

FIG. 10 is a section view of a portion of a linkage assembly in accordance with one embodiment of a present invention.

FIGS. 11A and 11B are section views of links in accordance with one embodiment of a present invention.

FIGS. 11C and 11D are section views of links in accordance with one embodiment of a present invention.

FIGS. 12A and 12B are section views of links in accordance with one embodiment of a present invention.

FIGS. 12C and 12D are section views of links in accordance with one embodiment of a present invention.

FIGS. 12E and 12F are section views of links in accordance with one embodiment of a present invention.

FIG. 13 perspective view of a portion of a cable in accordance with one embodiment of a present invention.

FIG. 14A is a plan view of a connector collar in accordance with one embodiment of a present invention.

FIG. 14B is another plan view of the connector collar illustrated in FIG. 14A.

FIG. 14C is a perspective view of the connector collar illustrated in FIG. 14A.

FIG. 15A is a section view of a connector inner cylinder in accordance with one embodiment of a present invention.

FIG. 15B is a plan view of the connector inner cylinder illustrated in FIG. 15A.

FIG. 15C is a perspective view of the connector inner cylinder illustrated in FIG. 15A.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

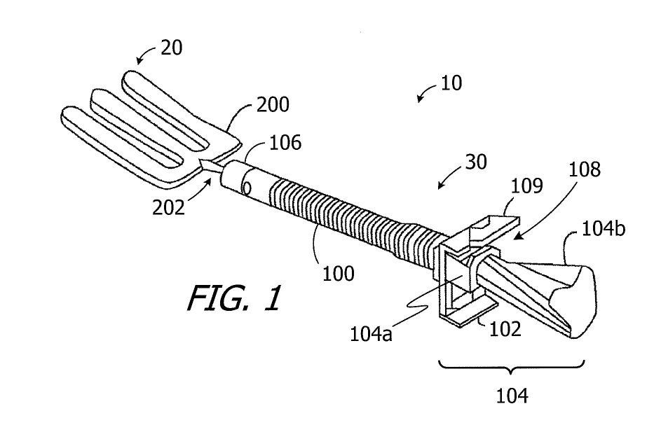

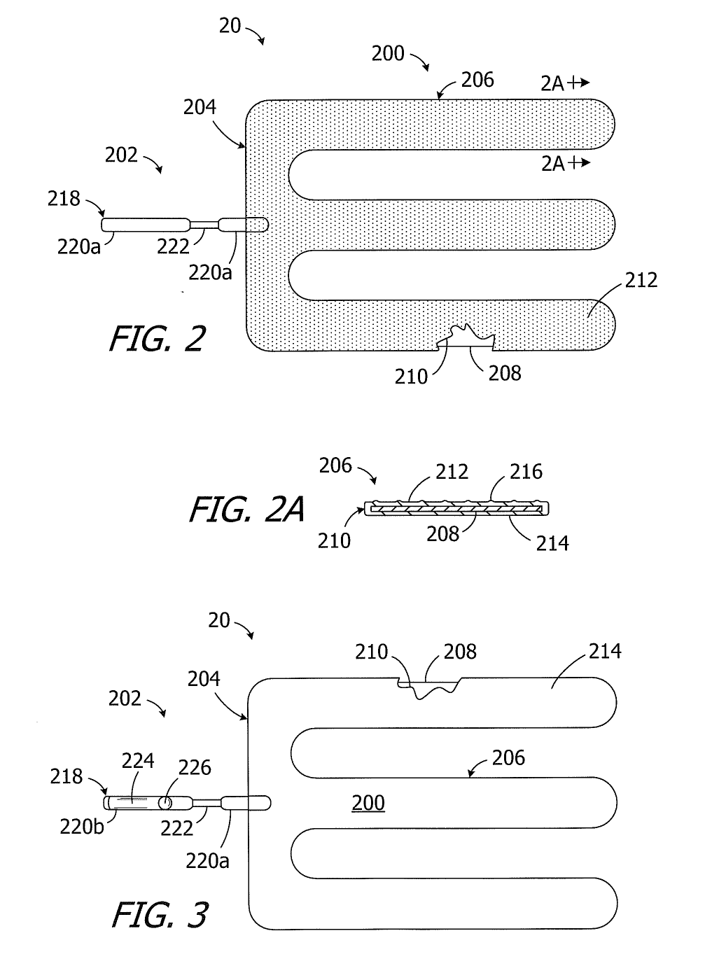

An exemplary surgical system in accordance with one embodiment of a present invention is generally represented by reference numeral 10 in FIG. 1. The surgical system includes a retractor apparatus 20 carried on a flexible articulating arm (or “arm”) 30. Exemplary retractor apparatus, such as apparatus 20, which may be releasably or permanently coupled to the arm 30, are discussed in greater detail below with reference to FIGS. 2-7. The exemplary arm 30 is discussed in greater detail below with reference to FIGS. 1 and 8-15C.

As illustrated for example in FIGS. 2-3, the exemplary retractor apparatus 20 includes a retractor 200 and a connector 202 that may be used to releasaby connect the retractor to, for example, the flexible articulating arm 30. The retractor 200 has a base 204 and a plurality of spaced members (or “fingers”) 206 that extend from the base. The embodiment illustrated in FIGS. 2-3 has three fingers 206 that are about 3 inches long, about 0.5 inch wide, and about 0.1 inch thick. Adjacent fingers 206 are separated by about 0.5 inch. The base 204 and the individual fingers 206 may be flat (as shown) or may be temporarily or permanently curved and/or bent at an angle relative to a flat plane, or relative to one another, much like a human hand. As such, the present retractor has a wide variety of surgical applications where it may take the place of a human hand and is especially useful in cardiac surgery. The present retractor apparatus are not limited to the illustrated “base and three finger” configuration and other exemplary configurations are described below with reference to FIGS. 6A-8.

The exemplary retractor 200 has a relatively hard inner portion 208, which provides structural stability, and a relatively soft outer portion 210, which makes the retractor atraumatic to tissue. The inner and outer portions are essentially the same overall shape, with the inner portion being slightly smaller. The relatively hard inner portion 208 may be formed from metal (e.g. stainless steel, annealed stainless steel or copper) or hard plastic. Suitable materials for the relatively soft outer portion 210 include, but are not limited to, relatively soft polymers such as silicone rubber or low durometer polyurethane.

In at least some embodiments, the retractor is provided with a relatively high friction outer surface, which will typically abut the retracted tissue during use, and a relatively low friction outer surface, which will typically face away from the retracted tissue during use. The additional friction associated with the relatively high friction outer surface may, for example, be the result of a surface geometry that makes the surface rough, yet atraumatic. Alternatively, the difference in friction between outer surfaces may stem from the use of different materials, material coatings and/or material treatments. The relatively high friction outer surface reduces the likelihood that the tissue being retracted will slide relative to the retractor during a surgical procedure when the tissue and retractor are wet, thereby increasing the likelihood that the retracted tissue will remain properly retracted. The relatively low friction outer surface allows objects, such as other tissue structures and the hands of the surgeon(s) and surgical assistants, to slide past the retractor. To that end, the coefficient of friction of the relatively high friction outer surface, when wet, may range from about 0.3 to 1.0 in some implementations, and may be about 0.4 in some implementations. The coefficient of friction of the relatively low friction outer surface, when wet, may range from about 0.05 to 0.3 in some implementations. It some implementations, coefficient of friction of the relatively high friction outer surface will be at least 50% higher than the coefficient of friction of the relatively low friction outer surface.

The relatively soft outer portion 208 of the retractor 200 illustrated in FIGS. 2-3, for example, has a relatively high friction outer surface 212 and a relatively low friction outer surface 214. Outer surfaces 212 and 214 are located on opposite sides of the retractor 200, and the sides together occupy the substantial majority of the retractor surface area. The relatively high friction outer surface 212 has a surface geometry defined by a plurality of bumps 216 (FIG. 2A) in the shape of partial spheres. The bumps 216 increase the surface area in contact with tissue. Although the bumps 216 are atraumatic, they increase frictional force on wet tissue surfaces through the increase in surface area. The bumps 216 may cover the entire associated side of the retractor (as shown) or a portion thereof. Additionally, the combination of a relatively high friction outer surface on one side of a retractor and a relatively low friction outer surface on the other side may also be employed in retractor apparatus that do not include a relatively soft outer portion, as is discussed below.

It should be noted here that in some retractor apparatus implementations, the retractor may lack a relatively soft outer portion and simply be formed from a relatively hard biocompatible metal (e.g. stainless steel or annealed stainless steel) or a relatively hard biocompatible plastic. Such retractors may (or may not) be configured with the combination of a relatively high friction outer surface and a relatively low friction outer surface, such as those illustrated in FIGS. 2-3. In those instances where a relatively high friction outer surface is present, the structures that define the surface geometry (e.g. bumps) may simply be formed in or on the relatively hard material.

The retractors described above and below may also be malleable, i.e. the retractor may be configured such that it can be readily bent by the physician to a desired shape, without springing back when released, and will remain in that shape during the surgical procedure. The stiffness of a malleable retractor must be low enough to allow the retractor to be bent, but high enough to resist bending when the forces associated with a surgical procedure are applied to the retractor. The present retractors may also be rigid, i.e. formed in a pre-set shape suitable for a particular application or formed in a pre-set shape that is suitable for a variety of applications. The present retractors may also be configured with rigid and malleable portions. With respect to numerical quantification, a malleable structure that is three inches in length would have a bending modulus between approximately 3 lb.-in.² and approximately 50 lb.-in.². It should be noted that the bending modulus range discussed here is primarily associated with initial deflection. In other words, the bending modulus range is based on the amount of force, applied at and normal to the free of the longitudinal axis of the structure, that is needed to produce 1 inch of deflection from an at rest (or no deflection) position.

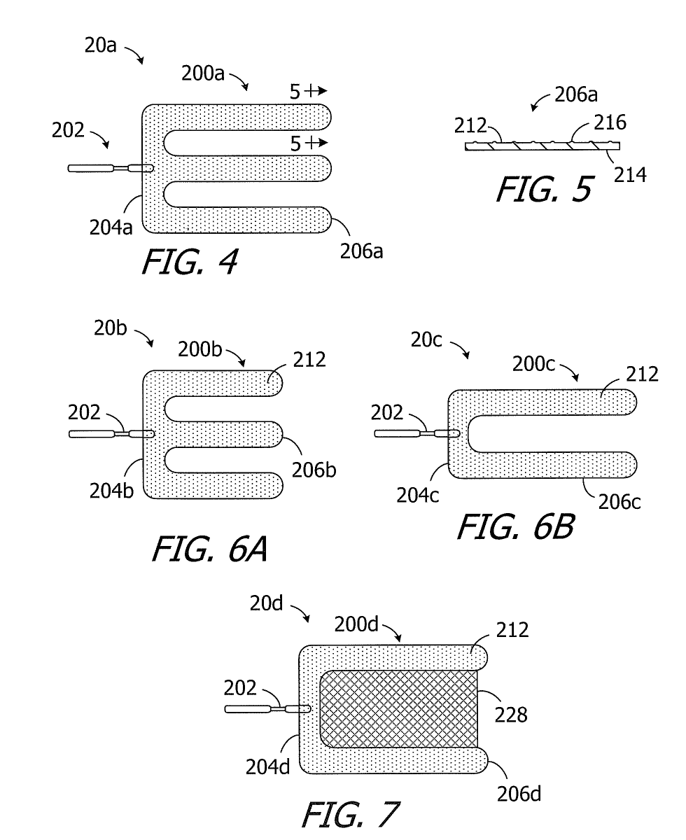

A malleable retractor may be constructed in a variety of ways. For example, in the exemplary two-portion retractor 200 illustrated in FIGS. 2-3, the inner portion 208 may be formed from a malleable metal such as copper or annealed stainless steel. Malleable retractors may, alternatively, be a unitary structure. For example, a retractor may formed from a malleable biocompatible metal, such as annealed stainless steel, without a relatively soft outer portion. As noted above, a retractor without a relatively soft outer portion may (or may not) be configured with a relatively high friction outer surface on one side and a relatively low friction outer surface on the other. Malleable plastics may also be employed. Referring to FIGS. 4 and 5, the exemplary retractor apparatus 20a includes a retractor 200a and a connector 202. The retractor 200a is substantially similar to the retractor 200 and similar elements are represented by similar reference numerals. To that end, the retractor 200a includes a base 204a, a plurality of fingers 206a, a relatively high friction outer surface 212 (with bumps 216) and a relatively low friction outer surface 214. Here, however, the base 204a and fingers 206a are formed from a malleable plastic/metal composite that is molded into the illustrated configuration. In other implementations, a relatively soft outer portion (with or without a relatively high friction surface on one side) may be formed over a malleable plastic/metal composite inner portion.

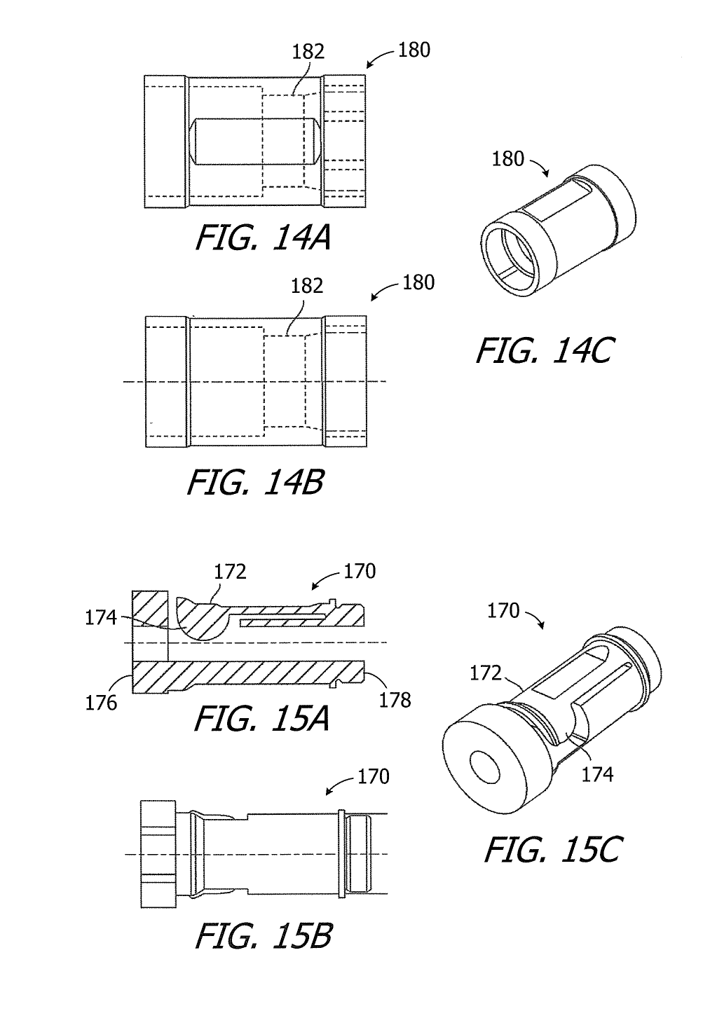

Turning back to FIGS. 1-3, the connector 202 that releasably secures the retractor apparatus 20 to the associated flexible articulating arm 30 may be any connector that is suitable for use with the corresponding connector 106 (discussed below) on the arm. In the illustrated embodiments, the connector 202 includes a shaft 218 with first and second end portions 220a and 220b connected to one another by an intermediate portion 222. The outer diameter of the intermediate portion 222 is less than that of the end portions 220a and 220b to enable the user to angle the retractor relative to the connector 106 while maintaining a stable connection to the arm. The first end portion 220a is secured to the retractor 200 and a portion of the first end portion is covered by the relatively soft outer portion 210. The second end portion 220b includes a channel 224 and a spherical indentation 226 that cooperate with the connector 106 in the manner described below with reference to FIGS. 14A-15C to allow the retractor apparatus to be easily secured to, and removed from, the arm by hand during the course of normal use.

Put another way, the connector 202 is one example of a structure which performs the function releasably securing a retractor to a corresponding connector on an arm. Other exemplary structures which perform the function of releasably securing a retractor to an arm include, but are not limited to, the following. A quick-connect, which is configured to be releasably connected to a corresponding structure (e.g. a cylindrical shaft) on the arm, may be provided on the retractor apparatus. Alternatively, the arm may be provided with the quick-connect and the retractor apparatus may be provided with a corresponding structure (e.g. a cylindrical shaft). In either case, the quick-connect may be configured such that the quick-connect collar slides distally or proximally to engage the post. The retractor apparatus may be provided with a male (or female) threaded connector and the arm may be provided with a corresponding female (or male) threaded connector. The retractor apparatus and/or the arm may be provided with a magnetic connector. The retractor apparatus may be provided with a ball that is configured to be received by a collet on the arm, or the arm may be provided with a ball that is configured to be received by a collet on the retractor apparatus. In either case, a cable or a rod may be used to retract the collet into the collar. The arm (or retractor apparatus) may be provided with a hollow cylinder and set screw arrangement and the retractor apparatus (or arm) may be provided with a shaft that is received within the cylinder. The arm (or retractor apparatus) may be provided with a hollow cylinder that has one or more internal indentations and the retractor apparatus (or arm) may be provided with a shaft that has one or more outwardly biased depressible members that fit into the indentations. The arm (or retractor apparatus) may be provided with a chuck and the retractor apparatus (or arm) may be provided with a shaft that is received within the chuck. The retractor apparatus (or arm) may be provided with a shaft including one or more transverse notches and the arm (or retractor apparatus) may be provided with a hollow cylinder that has one or more transverse holes. After the shaft is inserted into the hollow cylinder such that the notches are aligned with the holes, pins may be placed in the holes to prevent the shaft from moving.

The retractors described above and below may, in other implementations, be a permanent part of a surgical system such as, for example, surgical systems that include a flexible articulating arm. Here, the retractor will be permanently connected to the arm through the use of instrumentalities, such as adhesive, weld(s), and/or screws or other mechanical fasteners, that do not allow the retractor to be removed without disassembly or destruction of at least that portion of the system.

As noted above, the present retractor apparatus are not limited to the retractor configuration illustrated in FIGS. 2-3. The retractor apparatus generally represented by reference numeral 20b in FIG. 6A, for example, is substantially similar to the retractor apparatus 20 and similar elements are represented by similar reference numerals. To that end, the apparatus has a retractor 200b, with a base 204b and three fingers 206b, and a connector 202. Here, however, the fingers 206b are relatively short, as compared to fingers 206. The retractor 200b may also have the combination of a relatively high friction outer surface 212 on one side and a relatively low friction outer surface (not shown) on the opposite side. In various embodiments of the retractor apparatus 20b, the retractor 200b may be rigid or malleable, and the retractor may or may not include a relatively soft outer portion, as described above.

The present retractor apparatus are not limited to retractors with three fingers and, instead, may include few than three or more than three depending on the intended application. The retractor apparatus 20c illustrated in FIG. 6B, for example, is substantially similar to the retractor apparatus 20 and similar elements are represented by similar reference numerals. To that end, the apparatus has a retractor 200c, with a base 204c and fingers 206c, and a connector 202. Here, however, there are two fingers. The retractor 200c may also have a relatively high friction outer surface 212 on one side in combination with a relatively low friction outer surface (not shown) on the opposite side. In various embodiments of the retractor apparatus 20c, the retractor 200c may be rigid or malleable, and the retractor may or may not include a relatively soft outer portion, as described above.

It should also be noted that the fingers of a retractor may all be same length, such as is the case in the retractor 200b (FIG. 6A), or may have fingers of different length.

Another exemplary retractor apparatus is generally represented by reference numeral 20d in FIG. 7 is in some ways similar to the retractor apparatus 20 and similar elements are represented by similar reference numerals. For example, the retractor apparatus 20d has a retractor 200d, with a base 204d and a pair fingers 206d, and a connector 202. The retractor apparatus 20d also includes a mesh structure 228 that extends from one finger 206d to the other and from the base 204d to (or near) the free ends of the fingers. The mesh structure 228, which may be formed from silicone, nylon or any other biocompatible fabric or polymer, increases the effective surface area in contact with the retracted tissue and, therefore, provides more reliable retraction. The mesh structure is also provides more atraumatic retraction, which is especially useful when retracting more delicate tissue (e.g. lung tissue). The retractor 200d may also have the combination of a relatively high friction outer surface 212 on one side and a relatively low friction outer surface (not shown) on the opposite side. In various embodiments of the retractor apparatus 20d, the retractor 200d may be rigid or malleable, and the retractor may or may not include a relatively soft outer portion, as described above.

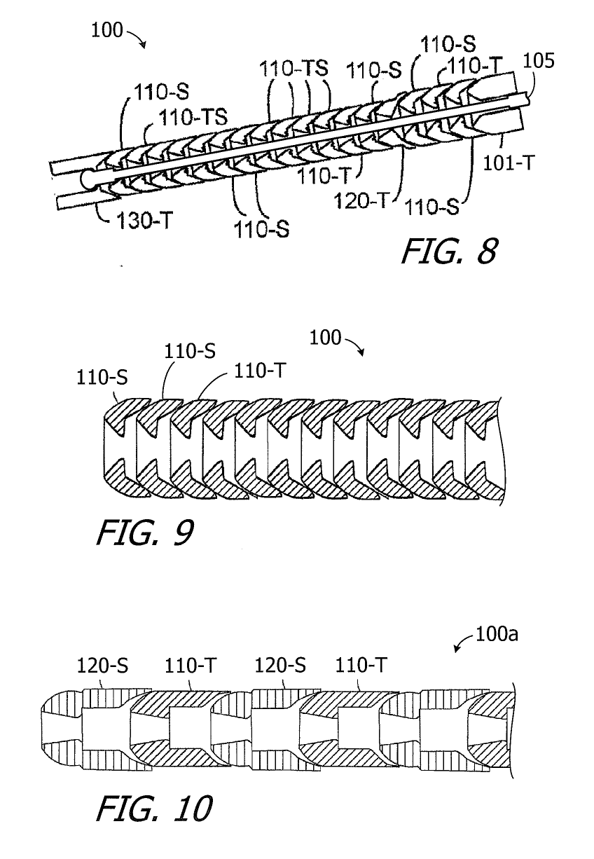

With respect to the other aspects of the exemplary surgical system 10 illustrated in FIG. 1, the flexible articulating arm 30 includes a linkage assembly 100, a C-bracket 102 that mounts the arm to the supporting structure (e.g. the side rail of an operating table), a tension block 104 that applies tension to the linkage assembly cable 105 (FIG. 8), and a connector 106 that releasably couples the retractor 20 to the arm. The tension block 104 includes a mounting block 104a and a rotatable handle 104b. The mounting block 104a may have an internal passage receiving a screw and, affixed to the screw, a transverse pin riding in slots formed in opposite sides of the mounting block. The pin and slots prevents the screw from rotating relative to mounting block 104a. The threads of the screw engage internal threads in the rotatable handle 104b, which also has an internal shoulder that can engage with the screw's head. The screw is directly attached (or otherwise operably connected to) the cable 105 and, accordingly, the handle 104b may be rotated to selectively increase or decrease the tension on the linkage assembly 100 to fix the orientation of the arm or permit repositioning of the arm. The C-bracket 102 and mounting block 104a may also be used to fix the location of the flexible articulating arm 30 on the supporting structure. To that end, a screw mechanism 108, including a pivot handle 109, may be used to drive the mounting block 104a towards the C-bracket 102.

Turning to FIGS. 8 and 9, the exemplary linkage assembly 100 includes a number of differently shaped links 101, 110, 120 and 130. Each linkage shape includes at least one contact surface, which contact couples to a neighboring contact surface of another link. Links 101 and 130 each have exactly one contact surface. The contact surface of link 101 is convex, while the contact surface of link 130 is concave. Links 110 and 120 each have two contact surfaces, one concave and the other convex. At one longitudinal end of the linkage assembly 100, link 130 is coupled with a link 110, while link 101 is coupled with a link 110 at the other longitudinal end. The tension cable 105 extends through the links and is anchored within link 130. An alternative linkage assembly 100a is illustrated in FIG. 10 and described in greater detail below.

The exemplary links may be formed from various metals and/or combinations thereof and the reference characters associated with each link include a material indicator. More specifically, a “-T” indicates that a link is composed primarily of titanium and a “-S” indicates that a link is composed primarily of stainless steel. With respect to links that employ two or more distinct metallic compounds, e.g. one for each contact surface, a “-TS” indicates that a link has a concave surface primarily composed of a titanium alloy, and a convex surface primarily composed of a stainless steel alloy, while a “-ST” indicates that a link has a concave surface primarily composed of a stainless steel alloy, and a convex surface primarily composed of a titanium alloy.

In the exemplary linkage assembly 100 illustrated in FIGS. 8 and 9, the concave and convex surfaces of the exemplary links 101, 110, 120 and 130 embody shapes, which for their materials, maximize static friction as well as kinetic friction when contacting each other under tension. In some implementations, a first link with a first contact surface (e.g. link 110-T) is composed of a first contact material and a second link with a second contact surface (e.g. link 110-S) is composed of a second contact material, with each of the contact materials primarily composed of a different metallic compound. A high friction coupling between the first link and the second link may created by the first contact surface contacting the second contact surface when induced by the tension cable 105. The first contact surface, composed of the first contact material, contacting the second contact surface, composed of the second contact material, has a higher friction coefficient than results from composing both contact surfaces of either contact material. Suitable friction coefficients may range from, but are not limited to, 0.3 to 0.3875.

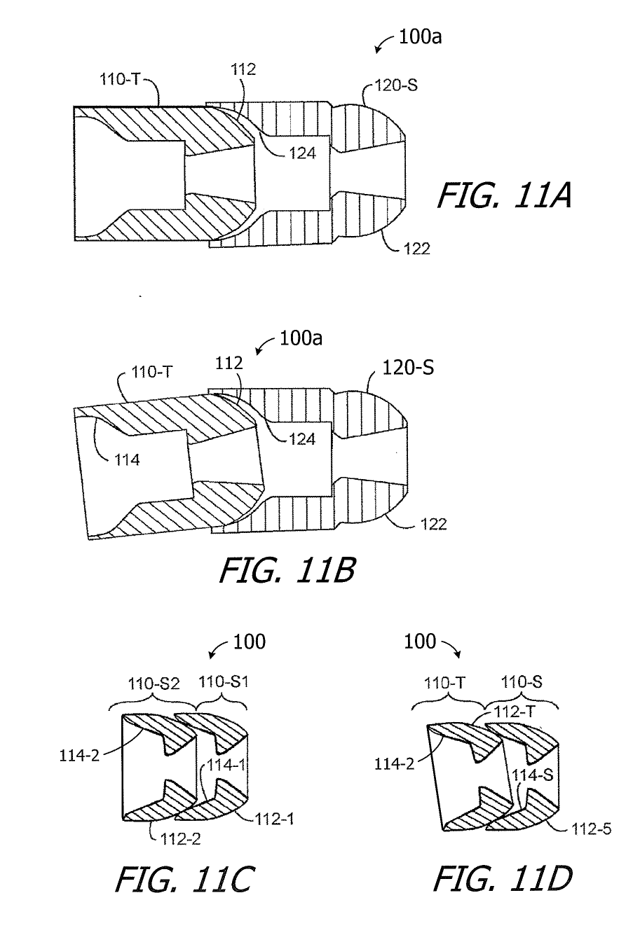

Turning to FIGS. 11A-11B, in the linkage assembly 100a illustrated in FIG. 10, at least two of the links (i.e. links 100-T and 120-S) are coupled through a spherical convex surface contacting a spherical concave surface. The spherical convex surface 112 connects with the semi-spherical concave surface 124. The diameters of the two surfaces are preferably slightly different, with the convex semi-spherical 112 diameter being larger than the semi-spherical diameter of the interfacing concave surface 124. Convex surface 112 and concave surface 124 form an interference fit when the two surfaces contact each other under tension. The wall of link 120-S is sufficiently thin and resilient where the two surfaces come together to provide an area contact between the links.

FIG. 11C shows two stainless steel links (labeled 110-S1 and 110-S2) from the exemplary linkage assembly illustrated in FIG. 10 coupled with a spherical convex surface contacting a conical concave surface. More specifically, the spherical convex surface 112-2 connects with the conical concave surface 114-1. The diameters of the two surfaces are slightly different, with the convex semi-spherical 112-2 diameter being larger than the conical diameter of the interfacing concave surface 114-1. Convex surface 112-2 and concave surface 114-1 form an interference fit when the two surfaces contact each other under tension. The wall of link 110-S1 is sufficiently thin and resilient where the two surfaces come together to provide an area of contact.

In FIG. 11D, links 110-T and 110-S from the exemplary linkage assembly illustrated in FIG. 10 form a coupling where a spherical convex titanium surface contacts a conical concave stainless steel surface, i.e. the spherical convex surface 112-T connects with the conical concave surface 114-S. The diameters of the two surfaces are slightly different, with the convex semi-spherical 112-T diameter being larger than the conical diameter of the interfacing concave surface 114-S. Convex surface 112-T and concave surface 114-S form an interference fit when the two surfaces contact each other under tension. The wall of link 110-S1 is sufficiently thin and resilient where the two surfaces come together to provide an of area contact.

The circular edge of the opening of each link illustrated in FIGS. 11A-11D may be concentric with the center of the imaginary sphere in which the surface lies when the links are fully engaged with each other. The edge is rounded to avoid a sharp edge that could damage the tensioning cable. The rounded edge has a very small radius of curvature to maximize the contact area of the mating convex and concave surfaces. The fact that the edge is rounded instead of sharp has negligible effect on the contact area.

The diameters of the convex and mating concave link surfaces may vary over the length of the linkage assembly. This supports the need for increased strength and/or stiffness at the proximal end of the articulating arm near the tension block 104, where the applied mechanical moment is greatest. The joints at the proximal end of the arm are preferably larger in diameter. This increases their rotational inertia, or resistance to rotation, in addition to providing greater frictional contact area than smaller distal beads located furthest from tension block 104. The greatest load-bearing link is frequently the most proximal link.

This link may be sunk into the body of the articulating column providing a mechanical lock, prohibiting rotation of this link.

One potential mode of failure of a flexible articulating arm that is used repeatedly is cable failure. If the cable fails in an arm with a single uniform cable, nothing is left holding the links together. This allows the links to fall into the surgical field. A variety of factors are associated with the potential for cable failure. The cable (e.g. cable 105) is shortened during use to create compressive forces between adjacent links and rigidify the linkage assembly, which results in tensile fatigue forces being applied to the cable. Shear forces are applied to the strands in contact with the inner radius of the links. If these radii are small, they contact a finite area of the cable and act as a knife edge, greatly wearing a localized area of the cable as it slides over these edges. If the arm is forcefully moved when in the rigid state (when all the slack is already removed from the cable), large loads will stretch the cable strands and greatly accelerate failure.

Various portions of the links may be configured so as to reduce the likelihood of cable failure. For example, the radius of curvature of areas contacting the cable may be increased, as alluded to above. The bend radius of a linkage assembly may be selected based on the minimum radius of curvature permissible for the cable that will be used in conjunction with that linkage assembly. The shape of the adjacent links may be designed to provide a gentle contour creating the selected radius, thereby more evenly distributing the load to more of the cable strands and minimizing contact forces applied to the strands in contact with the links and any sharp edges thereof.

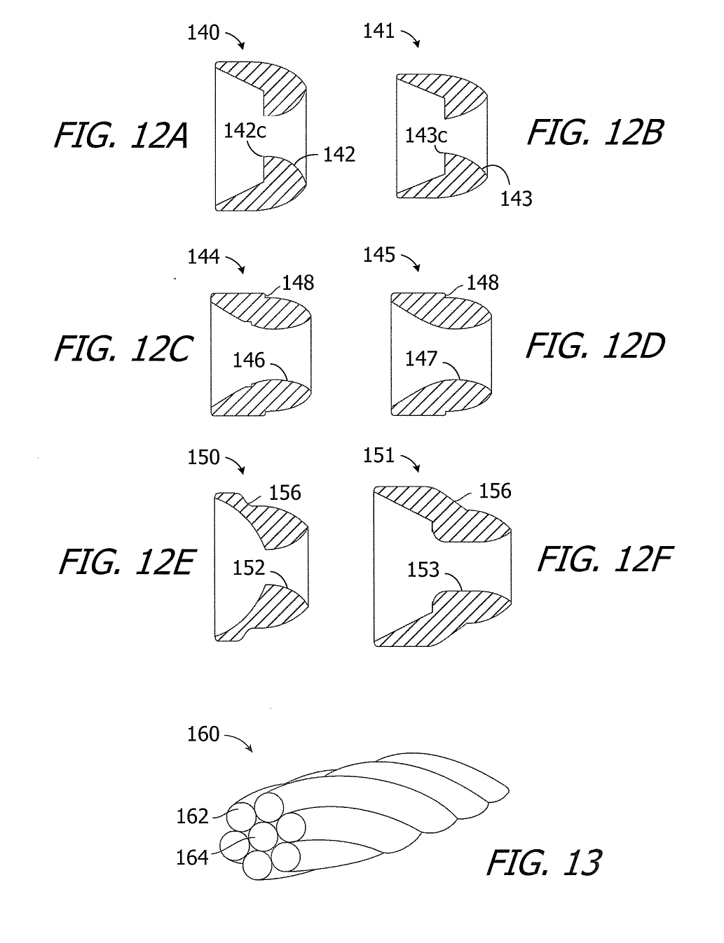

The links illustrated in FIGS. 12A-12F are examples of links that may be employed in the present linkage assemblies to reduce the likelihood of cable failure. Referring first to FIGS. 12A and 12B, links 140 and 141 include inner surfaces 142 and 143 that each have a relatively large radius of curvature. The inner surface corners 142c and 143c may also be rounded in some implementations. The links 144 and 145 illustrated in FIGS. 12C and 12D include inner surfaces 146 and 147 that each have a relatively large radius of curvature. The links 144 and 145 also have an external ridge 148 that prevents the arm assembly from bending beyond a preset limit. The links 150 and 151 illustrated in FIGS. 12E and 12F include inner surfaces 152 and 153 that each have a relatively large radius of curvature. The links 150 and 151 also have an external ridge 156 that prevents the arm assembly from bending beyond a preset limit. The external ridge 156 is more tapered than that illustrated in FIGS. 12C and 12D to provide a smoother external profile of the arm assembly.

Decreasing the coefficient of friction between cable and link contact surfaces also improves the life of the cable. A thin, biocompatible material may be used to provide a hard and lubricious surface. With no surface treatment, the cable may catch on the internal surface of the links causing large contact forces and strains on portions of the cable. The lubricious surface allows the cable to more easily slide along the surfaces of the links as tension is applied, thereby reducing the chance of larger point load or frictional wear on the cable. One option for the lubricious surface is hard chrome plating. The chrome is hard and lubricious, and thus serves as a good material for plating if the desired result is wear resistance. The links, the cable or both may be coated to provide this advantage.

In other implementations, the cable may include a device that will hold the links together despite cable failure. One example of such a cable is generally represented by reference numeral 160 in FIG. 13. The cable 160 includes a plurality of stainless steel strands 162 and at least one elastic (or superelastic) strand 164. When the strands 162 fail, the elastic nature of the strand 164 will cause that portion of the cable 160 to stretch and allow the flexible arm to fail while still holding the links together. One suitable material for the elastic strand 164 is a nickel titanium alloy sold under the trade name Nitinol.

With respect to the manner in which the retractors are releasably connected the flexible articulating arm 30 in the illustrated implementation, the exemplary connector 106 (FIG. 1) may be a two-part structure including the outer collar illustrated in FIGS. 14A-14C and the inner cylinder illustrated in FIGS. 15A-15C.

Referring first to FIGS. 15A-15C, the inner cylinder 170 includes a deflectable portion 172, which creates a spring effect, and a spherical surface 174 that is carried by the deflectable portion and is configured to slide along shaft channel 224 and mate with the shaft detent 226 (FIG. 3). Inner cylinder end 176 is secured to the associated arm, and the shaft 218 is inserted at end 178. The collar 180 is movable between a locked position which prevents movement of the shaft 218 and an unlocked position which permits withdrawal of the shaft, and is biased to the locked position by an internal coil spring (not shown). The collar 180 also includes a necked down portion 182. To insert the retractor shaft 218, collar 180 is moved away from cylinder end 176 until the collar 180 is in the unlock position where the neck down portion 182 does not apply force to the deflectable portion 172. After the shaft 218 is inserted and the spherical surface 174 of the deflectable portion 172 mates with the spherical concave detent 226, the collar 180 may be released. The spring (not shown) forces the collar 180 back to the lock position, where the neck down portion 182 comes into contact with the deflectable portion 172, forcing the spherical surface 174 to seat in the shaft detent 226, and locking the axial and rotational position of the retractor apparatus. Suitable materials for the inner cylinder 170 and collar 180 include stainless steel.

Additional details concerning the exemplary flexible articulating arms described above, as well as other arms, are provided in U.S. Pat. No. 6,860,668 and U.S. Patent Pub. No. 2005/0226682 A1, which are incorporated herein by reference.

Although the inventions disclosed herein have been described in terms of the preferred embodiments above, numerous modifications and/or additions to the above-described preferred embodiments would be readily apparent to one skilled in the art. By way of example, but not limitation, retractor apparatus may be provided with high friction surfaces on both sides. It is intended that the scope of the present inventions extend to all such modifications and/or additions and that the scope of the present inventions is limited solely by the claims set forth below.

I claim:

1. A retractor apparatus, comprising:
a malleable retractor; and
a connector secured to the malleable retractor and configured to secure the malleable retractor to a mechanical arm.

2. A retractor apparatus as claimed in claim 1, wherein the malleable retractor includes a base and a plurality of spaced members extending from the base.

3. A retractor apparatus as claimed in claim 2, further comprising:
a mesh structure between two of the spaced members.

4. A retractor apparatus as claimed in claim 1, wherein the malleable retractor includes a relatively hard inner portion and a relatively soft outer portion.

5. A retractor apparatus as claimed in claim 4, wherein the relatively hard inner portion is formed from a malleable metal or a malleable plastic/metal composite and the relatively soft outer portion is formed from a relatively soft polymer.

6. A retractor apparatus as claimed in claim 4, wherein
the malleable retractor defines first and second sides; and
the relatively soft outer portion has a relatively high friction outer surface associated with the first side and a relatively low friction outer surface associated with the second side.

7. A retractor apparatus as claimed in claim 1, wherein the malleable retractor is formed from a malleable metal or a malleable plastic/metal composite.

8. A retractor apparatus as claimed in claim 1, wherein
the malleable retractor defines first and second sides; and
the malleable retractor includes a relatively high friction outer surface associated with the first side and a relatively low friction outer surface associated with the second side.

9. A retractor apparatus as claimed in claim 1, wherein the connector is configured to releasably secure the retractor to the mechanical arm.

10. A retractor apparatus as claimed in claim 9, wherein the connector includes a shaft with a spherical indentation.

11. A retractor apparatus, comprising:
a retractor including a relatively hard inner portion and a relatively soft outer portion; and
a connector secured to the retractor and configured to secure the retractor to a mechanical arm.

12. A retractor apparatus as claimed in claim 11, wherein the retractor includes a base and a plurality of spaced members extending from the base.

13. A retractor apparatus as claimed in claim 12, further comprising:
a mesh structure between two of the spaced members.

14. A retractor apparatus as claimed in claim 11, wherein the relatively hard inner portion is formed from a metal, a plastic or a plastic/metal composite, and the relatively soft outer portion is formed from a relatively soft polymer.

15. A retractor apparatus as claimed in claim 11, wherein
the retractor defines first and second sides; and
the relatively soft outer portion has a relatively high friction outer surface associated with the first side and a relatively low friction outer surface associated with the second side.

16. A retractor apparatus as claimed in claim 11, wherein the connector is configured to releasably secure the retractor to the mechanical arm.

17. A retractor apparatus as claimed in claim 16, wherein the connector includes a shaft with a spherical indentation.

18. A surgical system, comprising:
an arm; and
a retractor apparatus, operably connected to the arm, including a malleable retractor.

19. A surgical system as claimed in claim 18, wherein the arm comprises a flexible articulating arm.

20. A surgical system as claimed in claim 19, wherein the flexible articulating arm includes a plurality of links and a tension cable.

21. A surgical system as claimed in claim 18, wherein
the arm includes a first connector;
the retractor apparatus includes a second connector; and
the first and second connectors are configured to releasably connect the retractor apparatus to the arm.

22. A surgical system as claimed in claim 18, wherein the malleable retractor includes a base and a plurality of spaced members extending from the base.

23. A surgical system as claimed in claim 22, further comprising:
a mesh structure between two of the spaced members.

24. A surgical system as claimed in claim 18, wherein the malleable retractor includes a relatively hard inner portion and a relatively soft outer portion.

25. A surgical system as claimed in claim 24, wherein the relatively hard inner portion is formed from a malleable metal or a malleable plastic/metal composite and the relatively soft outer portion is formed from a relatively soft polymer.

26. A surgical system as claimed in claim 24, wherein
the malleable retractor defines first and second sides; and
the relatively soft outer portion has a relatively high friction outer surface associated with the first side and a relatively low friction outer surface associated with the second side.

27. A surgical system as claimed in claim 18, wherein the malleable retractor is formed from a malleable metal or a malleable plastic/metal composite.

28. A surgical system as claimed in claim 18, wherein
the malleable retractor defines first and second sides; and
the malleable retractor includes a relatively high friction outer surface associated with the first side and a relatively low friction outer surface associated with the second side.

29. A surgical system, comprising:
an arm; and
a retractor apparatus, operably connected to the arm, including a relatively hard inner portion and a relatively soft outer portion.

30. A surgical system as claimed in claim 29, wherein the arm comprises a flexible articulating arm.

31. A surgical system as claimed in claim 30, wherein the flexible articulating arm includes a plurality of links and a tension cable.

32. A surgical system as claimed in claim 29, wherein
the arm includes a first connector;
the retractor apparatus includes a second connector; and
the first and second connectors are configured to releasably connect the retractor apparatus to the arm.

33. A surgical system as claimed in claim 32, wherein the retractor includes a base and a plurality of spaced members extending from the base.

34. A surgical system as claimed in claim 33, further comprising:
a mesh structure between two of the spaced members.

35. A surgical system as claimed in claim 32, wherein the relatively hard inner portion is formed from a metal, a plastic or a plastic/metal composite, and the relatively soft outer portion is formed from a relatively soft polymer.

36. A surgical system as claimed in claim 32, wherein
the retractor defines first and second sides; and
the relatively soft outer portion has a relatively high friction outer surface associated with the first side and a relatively low friction outer surface associated with the second side.

37. A retractor apparatus, comprising:
a retractor defining first and second sides and including a relatively high friction outer surface associated with the first side and a relatively low friction outer surface associated with the second side; and
a connector secured to the retractor and configured to secure the retractor to a mechanical arm.

38. A retractor apparatus as claimed in claim 37, wherein the retractor includes a base and a plurality of spaced members extending from the base.

39. A retractor apparatus as claimed in claim 38, further comprising:
a mesh structure between two of the spaced members.

40. A retractor apparatus as claimed in claim 37, wherein the retractor includes a relatively hard inner portion and a relatively soft outer portion; and
the relatively high friction outer surface and the relatively low friction outer surface are associated with the relatively soft outer portion.

41. A retractor apparatus as claimed in claim 40, wherein
the relatively hard inner portion is formed from a metal, a plastic or a plastic/metal composite, and the relatively soft outer portion is formed from a relatively soft polymer.

42. A retractor apparatus as claimed in claim 37, wherein the connector is configured to releasably secure the retractor to the mechanical arm.

43. A retractor apparatus as claimed in claim 42, wherein the connector includes a shaft with a spherical indentation.

44. A surgical system, comprising:
an arm; and
a retractor apparatus, operably connected to the arm, defining first and second sides and including a relatively high friction outer surface associated with the first side and a relatively low friction outer surface associated with the second side.

45. A surgical system as claimed in claim 44, wherein the arm comprises a flexible articulating arm.

46. A surgical system as claimed in claim 45, wherein the flexible articulating arm includes a plurality of links and a tension cable.

47. A surgical system as claimed in claim 44, wherein
the arm includes a first connector;
the retractor apparatus includes a second connector; and
the first and second connectors are configured to releasably connect the retractor apparatus to the arm.

48. A surgical system as claimed in claim 44, wherein the retractor includes a base and a plurality of spaced members extending from the base.

49. A surgical system as claimed in claim 48, further comprising:
a mesh structure between two of the spaced members.

50. A surgical system as claimed in claim 44, wherein
the retractor includes a relatively hard inner portion and a relatively soft outer portion; and
the relatively high friction outer surface and the relatively low friction outer surface are associated with the relatively soft outer portion.

51. A surgical system as claimed in claim 50, wherein
the relatively hard inner portion is formed from a metal, a plastic or a plastic/metal composite, and the relatively soft outer portion is formed from a relatively soft polymer.
